(19) United States
(12) Patent Application Publication
(10) Pub. No.: US 2003/0203355 A1
(43) Pub. Date: Oct. 30, 2003

(54) Fluorobodies: binding ligands with intrinsic fluorescence

(75) Inventors: Andrew M. Bradbury, Santa Fe, NM (US); Ahmet Zeytun, Los Alamos, NM (US); Geoffrey S. Waldo, Santa Fe, NM (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Los Alamos National Laboratory, Los Alamos, NM (US); The Regents of the University of California, Oakland, CA (US)

(21) Appl. No.: 10/132,067

(22) Filed: Apr. 24, 2002

(51) Int. Cl. C12Q 1/70; C12Q 1/68; G01N 33/53; C07K 14/435

(52) U.S. Cl. 435/5; 435/6; 435/7.1; 530/350

(57) ABSTRACT

The current invention provides binding ligands with intrinsic fluorescent activity. The invention also provides libraries of such binding ligands, methods of preparing such binding ligands and libraries, and methods of identifying a binding ligand that specifically binds to a target molecule.

### STATEMENT AS TO RIGHTS TO INVENTIONS MADE UNDER FEDERALLY SPONSORED RESEARCH OR DEVELOPMENT

This invention was made with government support under grant number DE-FG02-98ER62647 from the United States Department of Energy and Contract No. W-7405-ENG-36 awarded by the United States Department of Energy to The Regents of The University of CAlifornia. The government has certain rights in this invention.

### BACKGROUND OF THE INVENTION

Molecular diversity libraries with billions of different members have proved to be rich sources of binding ligands. Peptides and antibody libraries have been commonly used, but other ligands such as CTLA4, lipocalins, protein A, isolated light or heavy chain variable regions have also been displayed on the surface of filamentous phage within the context of large libraries from which high affinity binding ligands have been selected. Detection of these ligands, however, require the use of tags or secondary binding reagents. A binding ligand that has an intrinsic detection capability, e.g., fluorescence, would provide substantial benefits, for example, conferring the ability to monitor binding in real time.

Fluorescent proteins, e.g., green fluorescent protein (GFP) are intrinsically fluorescent proteins that are ideal candidates for such generating such ligands. However, although GFP has been displayed on the surface of bacteria, no fluorescent protein-based libraries have a been created or used in binding selection experiments. Attempts to insert linkers or random peptides within GFP have been unsuccessful, with most insertions rendering the GFP either non- or weakly fluorescent. One report described the identification of GFP-loop inserted peptide sequences with apparent nuclear localization activity ((Peelle, et al., Chem. Biol. 8:521-534, 2001), but at very high cytoplasmic GFP concentrations.

Other reports describe the use of GFP as a potential optical signaling protein, with GFP fluorescence (or FRET) modulated by changes in voltage, β-lactamase inhibitory protein concentration, calcium ions, zinc ions or pH. Furthermore, fluorescent GFP constructs containing insertions with the potential to measure changes in phosphorylation, protease activity, glutamate concentration and redox potential have also been referred to.

In general, the environmental modification of GFP fluorescence is mediated by the insertion of additional protein domains within the GFP sequence, with all but one of such modified GFPs having insertions in a single position, either tyrosine 145 or the equivalent of tyrosine 145 after circular permutation.

Current methods to detect targets using binding ligands, e.g., antibodies, require the use of secondary detectors, such as secondary antibodies labeled with a detection moiety. The current invention provides binding ligands, such as GFP-based binding ligands, with intrinsic fluorescent affinity. Thus, these ligands offer advantages over existing technologies as they do not require the use of other reagents either coupled to the protein or added to the reaction mixture to detect binding. For example, the fluorescent binding ligands of the invention, also referred to herein as “fluorobodies”, can be used to directly detect binding in real time. In addition to being useful in all applications for which antibodies, or antibody fragments, are currently used (e.g., immunofluorescence, immunohistochemistry, immunoprecipitation, western blotting, ELISAs, inhibition assays and protein-protein interaction studoes), fluorobodies can also be used in novel applications for which antibodies or antibody fragments are less suitable. Such applications include protein arrays, high throughput drug screening and biosensors.

### BRIEF SUMMARY OF THE INVENTION

The current invention provides binding ligands with intrinsic fluorescence, libraries of these ligands, and methods of preparing the ligands. In one aspect, the invention provides a binding ligand with intrinsic fluorescence comprising a fluorescent protein that has a structure with a root mean square deviation of less than 5 angstroms from the 11 beta strands of the green fluorescent protein (GFP) structure MMDB Id: 5742; wherein the fluorescent protein comprises heterologous binding sites in at least two loop positions, often three or four loop positions, on the surface of the fluorescent protein; and the binding ligand has fluorescent activity. Typically, the fluorescent protein has increased folding ability in comparison to a protein having the sequence of SEQ ID NO:2 or SEQ ID NO:4.

Often, the loop positions of the binding ligand are on the same face of the protein. In one embodiment, the loop positions are within 5 amino acids of the positions selected from the group consisting of positions 9-11, 36-40, 81-83, 114-118, 154-160, and 188-199 as determined by maximal correspondence to SEQ ID NO:2. In another embodiment, the loop positions are within 5 amino acids of the positions selected from the group consisting of positions 23-24, 48-56, 101-103, 128-143, 172-173, and 213-214 as determined by maximal correspondence to SEQ ID NO:2.

Alternatively, the loop position are within 5 amino acids of the positions selected from the group consisting of positions 37-39, 75-81, 114-117, 153-156, 185-192 as determined by maximal correspondence to SEQ ID NO:4; or are within 5 amino acids of the positions selected from the group consisting of positions 22-26, 100-103, 167-170, and 204-209 as determined by maximal correspondence to SEQ ID NO:4.

The binding sites of a fluorescent binding ligand of the invention can comprise random peptides or can comprise complementarity determining regions (CDRs).

In one embodiment, binding ligand comprises a fluorescent protein having the sequence set forth in SEQ ID NO:5.

In another aspect, the invention provides an expression vector comprising a nucleic acid sequence encoding a fluorescent binding ligand as set forth above, additionally provides a host cell comprising the expression vector.

The invention also provides a library comprising a population of nucleic acid sequences encoding fluorescent binding ligands as set forth above. In some embodiments, the library comprises a nucleic acid sequence encoding a fluorescent binding ligand that is linked to a polypeptide selected from the group consisting of a phage coat polypeptide, a bacterial outer membrane protein, and a DNA binding protein.

The library can be any kind of library, for example a display library such as a phage display library, a ribosomal display library, an mRNA display library, a bacterial display library, or a yeast display library.

In another aspect, the invention provides a method of preparing a binding ligand with intrinsic fluorescence that binds to a target antigen, the method comprising providing a fluorescent protein that has a structure with a root mean square deviation of less than 5 angstroms from the 11 beta strands of the green fluorescent protein (GFP) structure MMDB Id: 5742; and inserting a heterologous binding site into at least two loop regions, often three or four loop regions, on the surface of the protein, thereby obtaining a binding ligand with intrinsic fluorescence.

In another aspect, the invention provides a method of identifying a binding ligand with intrinsic fluorescence that specifically binds to a target molecule, the method comprising: providing a library as set forth above; screening the library with the target molecule; and selecting a binding ligand that binds to the target molecule.

### BRIEF DESCRIPTION OF THE DRAWINGS

FIG. 1 depicts the structure of a GFP variant with enhanced folding activity (a “superfolder”).

FIG. 2 depicts the structure of the GFP superfolder and the sites of insertion of complementarity determining regions (CDRs)

FIG. 3a-e shows the results of screening of a library of GFP binding ligands generated using either random sequence or CDR insertions with five different antigens.

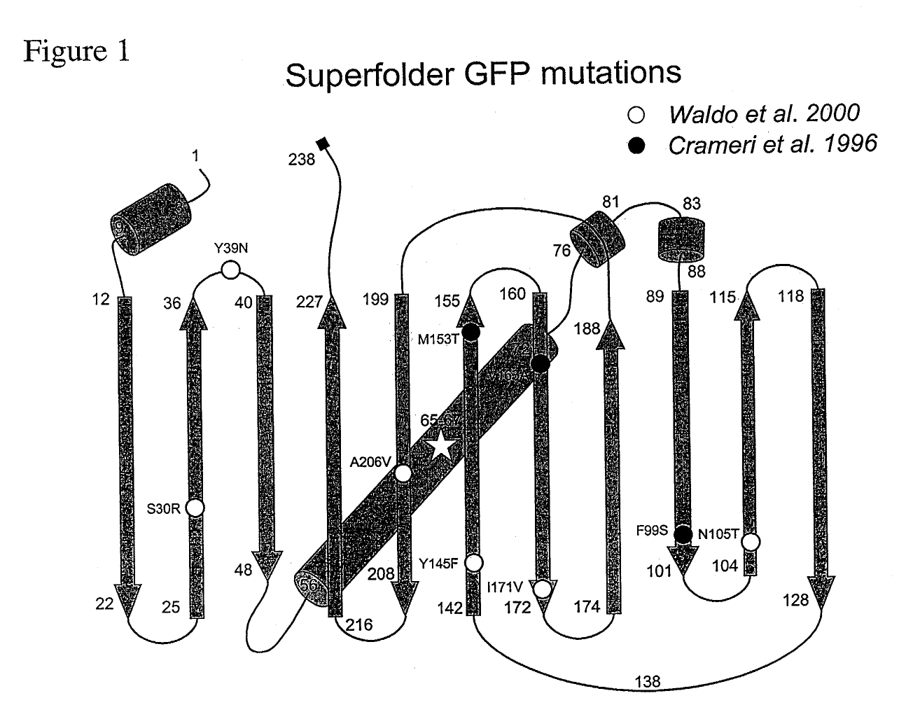

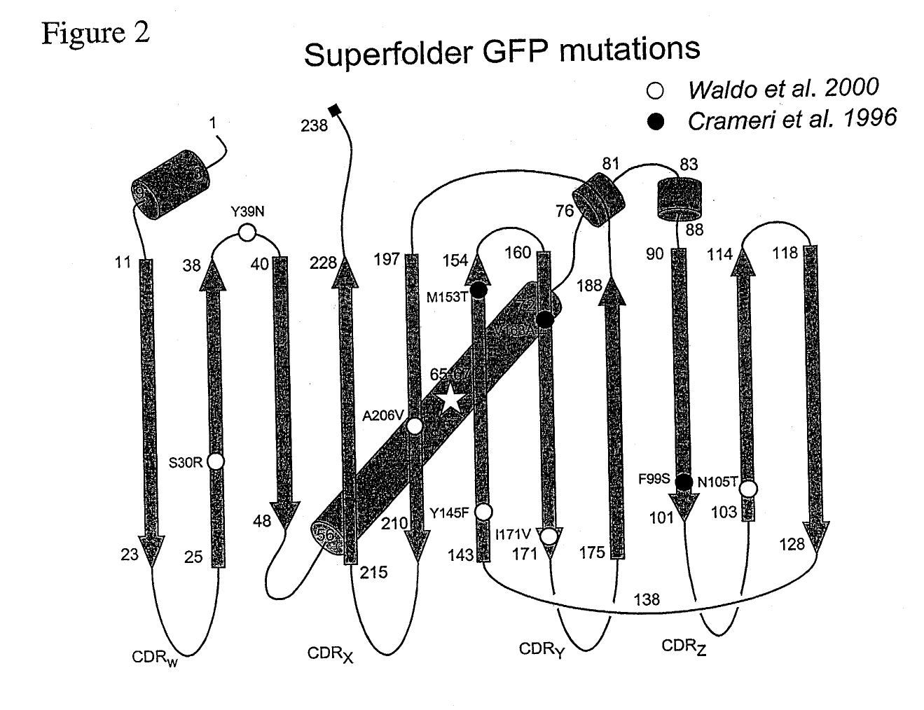

### DETAILED DESCRIPTION OF THE INVENTION

Definitions

The term “intrinsic fluorescence” as used herein refers to the ability of a compound to emit fluorescent light upon excitation with light of the appropriate wavelength.

A “fluorescent protein” as used herein is a protein that has intrinsic fluorescence. Typically, a fluorescent protein has a structure that includes an 11 strand beta barrel.

A “green fluorescent protein” as used herein refers to a polypeptide, or fluorescent fragments thereof, that: (1) have an amino acid sequence that has greater than about 65% amino acid sequence identity, 70%, 75%, 80%, 85%, 90%, preferably 91%, 92%, 93%, 94%, 95%, 96%, 97%, 98% or 99% or greater amino acid sequence identity, preferably over a window of at least about 25, 50, 100, 200 or more amino acids, to a GFP variant sequence (referred to herein as a “GFP folder”) as set forth in SEQ ID NO:2, or SEQ ID NO:8, (referred to herein as “wildtype GFP”); (2) bind to antibodies raised against an immunogen comprising an amino acid sequence of SEQ ID NO:2 or SEQ ID NO:8 and conservatively modified variants thereof; (3) is encoded by a nucleic acid that specifically hybridizes (with a size of at least about 100, preferably at least about 500 or more nucleotides) under stringent hybridization conditions to a sequence SEQ ID NO: 1 or SEQ ID NO:7 and conservatively modified variants thereof; or (4) is encoded by a nucleic acid sequence that has greater than about 95%, preferably greater than about 96%, 97%, 98%, 99%, or higher nucleotide sequence identity, preferably over a region of at least about 30, 50, 100, 200, 500, or more nucleotides, to SEQ ID NO: 1 or SEQ ID NO:7.

The “MMDB Id: 5742 structure” as used herein refers to the GFP structure disclosed by Ormo & Remington, MMDB Id: 5742, in the Molecular Modeling Database (MMDB), PDB Id: 1EMA PDB Authors: M.Ormo & S. J.Remington PDB Deposition: Aug. 1, 1996 PDB Class: Fluorescent Protein PDB Title: Green Fluorescent Protein From Aequorea Victoria. The Protein Data Bank (PDB) reference is Id PDB Id: 1EMA PDB Authors: M.Ormo & S. J.Remington PDB Deposition: Aug. 1, 1996 PDB Class: Fluorescent Protein PDB Title: Green Fluorescent Protein From Aequorea Victoria. (see, e.g., Ormo et al. “Crystal structure of the Aequorea victoria green fluorescent protein.” Science 1996 Sep 6;273(5280):1392-5; Yang et al, “The molecular structure of green fluorescent protein.” Nat Biotechnol. 1996 October;14(10):1246-51).

A “red fluorescent protein” or “dsRED” as used herein refers to a Discosoma sp. red fluorescent (dsRED) polypeptide, or fluorescent fragments thereof, that: (1) have an amino acid sequence that has greater than about 65% amino acid sequence identity, 70%, 75%, 80%, 85%, 90%, preferably 91%, 92%, 93%, 94%, 95%, 96%, 97%, 98% or 99% or greater amino acid sequence identity, preferably over a window of at least about 25, 50, 100, 200 or more amino acids, to a sequence of SEQ ID NO:4; (2) bind to antibodies raised against an immunogen comprising an amino acid sequence of SEQ ID NO:4 and conservatively modified variants thereof; (3) is encoded by a nucleic acid that specifically hybridizes (with a size of at least about 100, preferably at least about 500 or more nucleotides) under stringent hybridization conditions to a sequence SEQ ID NO:3 and conservatively modified variants thereof; or (4) is encoded by a nucleic acid sequence that has greater than about 95%, preferably greater than about 96%, 97%, 98%, 99%, or higher nucleotide sequence identity, preferably over a region of at least about 30, 50, 100, 200, 500, or more nucleotides, to SEQ ID NO:3.

“Root mean square deviation” (“RMSD”) refers to the root mean square superposition residual in Angstroms. This number is calculated after optimal superposition of two structures, as the square root of the mean square distances between equivalent C-alpha-atoms.

A “fluorescent binding ligand” (also referred to herein as a “fluorobody”) as used herein refers to a polypeptide that has intrinsic fluorescence activity and specifically binds to a binding partner via heterologous amino acid residues introduced into loop regions of a fluorescent protein, e.g., GFP. The fluorescent protein therefore serves as a “backbone” (or “scaffold” or “framework”) of the fluorescent binding ligand.

A “binding site” as used herein is an amino acid sequence inserted into a loop region that specifically binds a binding partner.

The term “heterologous” when used with reference to portions of a nucleic acid indicates that the nucleic acid comprises two or more subsequences that are not found in the same relationship to each other in nature. For instance, a nucleic acid is typically recombinantly produced, having two or more sequences from unrelated genes arranged to make a new functional nucleic acid, e.g., a nucleic acid encoding a fluorescent protein from one source and a nucleic acid encoding a peptide sequence from another source. Similarly, a heterologous protein indicates that the protein comprises two or more subsequences that are not found in the same relationship to each other in nature (e.g., a fusion protein).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a BLAST or BLAST 2.0 sequence comparison algorithms with default parameters described below, or by manual alignment and visual inspection. Such sequences are then said to be “substantially identical.” This definition also refers to the compliment of a test sequence. Preferably, the identity exists over a region that is at least about 22 amino acids or nucleotides in length, or more preferably over a region that is 30, 40, or 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. AppL. Math. 2:482 (1981), by the homology alignment algorithm of Needleman & Wunsch, J. Mol. Biol 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul etal., Nuc. Acids Res. 25:3389-3402 (1977) and Altschul et al., J. Mol. Biol. 215:403-410 (1990), respectively. BLAST and BLAST 2.0 are used, typically with the default parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “as determined by maximal correspondence” in the context of referring to a reference SEQ ID NO means that a sequence is maximally aligned with the reference SEQ ID NO over the length of the reference sequence using an algorithm such as BLAST set to the default parameters. Such a determination is easily made by one of skill in the art.

The term “link” as used herein refers to a physical linkage as well as linkage that occurs by virtue of co-existence within a biological particle, e.g., phage, bacteria, yeast or other eukaryotic cell.

“Physical linkage” refers to any method known in the art for functionally connecting two molecules, including without limitation, recombinant fusion with or without intervening domains, intein-mediated fusion, non-covalent association, covalent bonding (e.g., disulfide bonding and other covalent bonding), hydrogen bonding; electrostatic bonding; and conformational bonding, e.g., antibody-antigen, and biotin-avidin associations.

“Fused” refers to linkage by covalent bonding.

As used herein, “linker” or “spacer” refers to a molecule or group of molecules that connects two molecules, such as a fluorescent binding ligand and a display protein or nucleic acid, and serves to place the two molecules in a preferred configuration.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, N. Y. (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

The term “complementarity determining region” or “CDR” as used herein refers to the art-recognized term as exemplified by the Kabat and Chothia CDR definitions. CDRs are also generally known as hypervariable regions or hypervariable loops (Chothia and Lesk (1987) J. Mol. Biol. 196: 901; Chothia et al. (1989) Nature 342: 877; E. A. Kabat et al., Sequences of Proteins of Immunological Interest (National Institutes of Health, Bethesda, Md.) (1987); and Tramontano et al. (1990) J. Mol. Biol. 215: 175). Variable region domains typically comprise the amino-terminal approximately 105-115 amino acids of a naturally-occurring immunoglobulin chain (e.g., amino acids 1-110), although variable domains somewhat shorter or longer are also suitable for forming single-chain antibodies.

As used herein, “random peptide sequence” refers to an amino acid sequence composed of two or more amino acid monomers and constructed by a stochastic or random process. A random peptide can include framework or scaffolding protein sequences, e.g., GFP protein sequences, that may comprise invariant sequences.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “binding polypeptide” or “binding ligand” as used herein refers to a polypeptide that specifically binds to a target molecule (e.g. an antigen). Although a binding ligand may comprises a region from an immunoglobulin fragment, such as a CDR, binding polypeptides are typically distinguished from antibodies in that binding polypeptides do not have the same structural fold as immunoglobulins, or immunoglobulin fragments.

A “target molecule” in the context of this invention may be any molecule that will selectively bind to a fluorescent binding ligand of the invention. Typically, the target molecule is a protein, such as an antigen, or a receptor and the like, but may also be a non-protein molecule, e.g., a carbohydrate or lipid, haptens, organic molecules, small molecule pharmaceuticals, post-translational modifications occurring on polypeptides.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19: 5081; Ohtsuka et al. (1985) J. Biol. Chem. 260: 2605-2608; and Cassol et al (1992); Rossolini et al, (1994) Mol. Cell. Probes 8: 91-98). The term nucleic acid is used interchangeably with gene, cDNA, and mRNA encoded by a gene.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The terms “isolated” or “biologically pure” refer to material which is substantially or essentially free from components which normally accompany it as found in its native state. However, the term “isolated” is not intended refer to the components present in an electrophoretic gel or other separation medium. An isolated component is free from such separation media and in a form ready for use in another application or already in use in the new application/milieu.

As used herein “random peptide library” refers to a set of polynucleotide sequences that encodes a set of random peptides, and to the set of random peptides encoded by those polynucleotide sequences, as well as the fusion proteins containing those random peptides.

As used herein, “CDR library” refers to a set of polynucleotide sequences that encode CDR regions and to the set of CDR polypeptide sequences encoded by those polynucleotide sequences, as well as the fusion proteins containing the CDR sequences.

The phrase “specifically (or selectively) binds” to a binding partner, e.g., an antigen, or “specifically (or selectively) reactive with,” when referring to a protein or peptide, refers to a binding reaction that is determinative of the presence of the protein in a heterogeneous population of proteins and other biologics. Thus, under designated assay conditions, the specified antigen binds to a particular protein above background, e.g., at least two times the background, and does not substantially bind in a significant amount to other proteins present in the sample. Typically a specific or selective reaction will be at least twice background signal or noise and more typically more than 10 to 100 times background. Specific binding to an antibody under these conditions may require an antibody that is selected for its specificity for a particular protein. For example, polyclonal antibodies raised to a particular protein or antigen can be selected to obtain only those polyclonal antibodies that are specifically immunoreactive with the antigen, and not with other proteins, except for polymorphic variants, orthologs, and alleles of the protein. This selection may be achieved by subtracting out antibodies that cross-react with the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

The term “population” as used herein means a collection of components such as polynucleotides, portions or polynucleotides or proteins. A “mixed population: means a collection of components which belong to the same family of nucleic acids or proteins (i.e., are related) but which differ in their sequence (i.e., are not identical) and hence in their biological activity.

A “display vector” refers to a vector used to create a cell or virus that displays, i.e., expresses a display protein comprising a heterologous polypeptide, on its surface or in a cell compartment such that the polypeptide is accessible to test binding to target molecules of interest, such as antigens.

A “display library” refers to a population of display vehicles, often, but not always, cells or viruses. The “display vehicle” provides both the nucleic acid encoding a peptide as well as the peptide, such that the peptide is available for binding to a target molecule and further, provides a link between the peptide and the nucleic acid sequence that encodes the peptide. Various “display libraries” are known to those of skill in the art and include libraries such as phage, phagemids, yeast and other eukaryotic cells, bacterial display libraries, plasmid display libraries as well as in vitro libraries that do not require cells, for example ribosome display libraries or mRNA display libraries, where a physical linkage occurs between the mRNA or cDNA nucleic acid, and the protein encoded by the mRNA or cDNA.

A “phage expression vector” or “phagemid” refers to any phage-based recombinant expression system for the purpose of expressing a nucleic acid sequence in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

A “phage display library” refers to a “library” of bacteriophages on whose surface is expressed exogenous peptides or proteins. The foreign peptides or polypeptides are displayed on the phage capsid outer surface. The foreign peptide can be displayed as recombinant fusion proteins incorporated as part of a phage coat protein, as recombinant fusion proteins that are not normally phage coat proteins, but which are able to become incorporated into the capsid outer surface, or as proteins or peptides that become linked, covalently or not, to such proteins. This is accomplished by inserting an exogenous nucleic acid sequence into a nucleic acid that can be packaged into phage particles. Such exogenous nucleic acid sequences may be inserted, for example, into the coding sequence of a phage coat protein gene. If the foreign sequence is “in phase” the protein it encodes will be expressed as part of the coat protein. Thus, libraries of nucleic acid sequences, such as a genomic library from a specific cell or chromosome, can be so inserted into phages to create “phage libraries.” As peptides and proteins representative of those encoded for by the nucleic acid library are displayed by the phage, a “peptide-display library” is generated. While a variety of bacteriophages are used in such library constructions, typically, filamentous phage are used (Dunn (1996) Curr. Opin. Biotechnol. 7:547-553). See, e.g., description of phage display libraries, below.

The term “amplification” means that the number of copies of a polynucleotide is increased.

Fluorescent Proteins

A variety of fluorescent proteins can be used as “backbone” for insertion of peptide sequences to generate the fluorescent binding ligands of the invention. These include GFP and its variants, such as cyan fluorescent protein, blue fluorescent protein, yellow fluorescent proteins, etc. Typically, these variants share at least 65%, more often 80%, 90% or greater, sequence identity with SEQ ID NO:2 (or SEQ ID NO:8.)

Other fluorescent proteins, such as the red fluorescent protein dsRED (Matz et al., Nat. Biotechnol. 17:969-973, 1999), e.g., SEQ ID NO:4 (see, e.g., accession number AF168419 version AF168419.2), can also be used. dsRED is structurally similar to GFP, maintaining the 11 strand beta barrel structure of MMDB Id: 5742

Any fluorescent protein can be used that has a structure with a root mean square deviation of less than 5 angstroms, often less than 3, or 4 angstroms, and preferably less than 2 angstroms from the 11 strand beta barrel structure of MMDB Id:5742. As appreciated by one of ordinary skill in the art, such a suitable fluorescent protein structure can be identified using comparison methodology well known in the art. In identifying the protein, a crucial feature in the alignment and comparison to the MMDB ID:5742 structure is the conservation of the 11 beta strands, and the topology or connection order of the secondary structural elements (see, e.g., Ormo et al. “Crystal structure of the Aequorea Victoria green fluorescent protein.” Science 1996 Sep 6;273(5280):1392-5; Yang et al, “The molecular structure of green fluorescent protein.” Nat Biotechnol. 1996 Oct; 14(10):1246-51). Typically, most of the deviations between a fluorescent protein and the GFP strucuture are in the length(s) of the connecting strands or linkers between the crucial beta strands, see, e.g., the comparison of dsRed and GF (Yarbrough et al., Proc Natl Acad Sci USA 98:462-7, 2001). In Yarbrough et al., alignment of GFP and dsREDe is shown pictorially. From the stereo diagram, it is apparent that the 11 beta-strand barrel is rigorously conserved between the two structures. The c-alpha backbones are aligned to within 1 angstrom RMSD over 169 amino acids although the sequence identity is only 23% comparing dsRed and GFPtural superposition.

In comparing structure, the two structures to be compared are aligned using algorithms familiar to those with average skill in the art, using for example the CCP4 program suite. COLLABORATIVE COMPUTATIONAL PROJECT, NUMBER 4. 1994. “The CCP4 Suite: Programs for Protein Crystallography”. Acta Cryst. D50, 760-763. IN using such a program, the user inputs the PDB coordinate files of the two structures to be aligned, and the program generates output coordinates of the atoms of the aligned structures using a rigid body transformation (rotation and translation) to minimize the global differences in position of the atoms in the two structures. The output aligned coordinates for each structure can be visualized separately or as a superposition by readily-available molecular graphics programs such as RASMOL, Roger A. Sayle and E. J. Milner-White, “RasMol: Biomolecular graphics for all”, Trends in Biochemical Science (TIBS), September 1995, Vol. 20, No. 9, p.374.), or Swiss PDB Viewer, Guex, N and Peitsch, M. C.(1996) Swiss-PdbViewer: A Fast and Easy-to-use PDB Viewer for Macintosh and PC. Protein Data Bank Quaterly Newsletter 77, pp. 7.

In considering the RMSD, the RMSD value scales with the extent of the structural alignments and this size is taken into consideration when using the RMSD as a descriptor of overall structural similarity. The issue of scaling of RMSD is typically dealt with by including blocks of amino acids that are aligned within a certain threshold. The longer the unbroken block of aligned sequence that satisfies a specified criterion, the ‘better’ aligned the structures are. In the dsRed example, 164 of the c-alpha carbons can be aligned to within 1 angstrom of the GFP. Typically, users skilled in the art will select a program that can align the two trial structures based on rigid body transformations, for example DALI, Holm, L. & Sander, C. Protein-structure comparison by alignment of distance matrices. Journal of Molecular Biology 1993, 233, 123-138. The server site for the computer implementation of the algorithm is available, for example, at dali@ebi.ac.uk. The output of the DALI algorithm are blocks of sequence that can be superimposed between two structures using rigid body transformations. Regions with Z-scores at or above a threshold of Z=2 are reported as similar. For each such block, the overall RMSD is reported.

The RMSD of a fluorescent protein for use in the invention is within 5 angstroms for at least 80% of the sequence within the 11 beta strands. Preferably, RMSD is within 2 angstroms for at least 90% of the sequence within the 11 beta strands (the beta strands determined by visual inspection of the two aligned structures graphically drawn as superpositions, and comparison with the aligned blocks reported by DALI program output). As appreciated by one of skill in the art, the linkers between the beta strands can vary considerably, and need not be superimpossible between structures, since by definition replacement of such linker, e.g., by CDRs, retains the fluorescence of the protein, which is possible only if the beta barrel structure is preserved.

In preferred embodiments, the fluorescent protein is a mutated version of the protein or a variant of the protein that has improved folding properties or solubility in comparison to the protein. Often, such proteins can be identified, for example, using methods described in WO0123602 and other methods to select for increased folding.

For example, to obtain a fluorescent protein with increased folding ability, a “bait” or “guest” peptide that decreases the folding yield of the fluorescent protein is linked to the fluorescent protein. The guest peptide can be any peptide that, when inserted, decreases the folding yield of the fluorescent protein. A library of mutated fluorescent proteins is created. The bait peptide is inserted into the fluorescent protein and the degree of fluorescence of the protein is assayed. Those clones exhibit increased fluorescence relative to a fusion protein comprising the bait peptide and parent fluorescent protein are selected (the fluorescent intensity reflects the amount of properly folded fluorescent protein). The guest peptide may be linked to the fluorescent protein at an end, or may be inserted at an internal site.

The binding ligands with fluorescent activity of the invention are generated by the insertion of peptide sequences at the loop regions of a fluorescent protein. A loop sequence is defined as the solvent-exposed peptide sequence connecting two beta strands, a beta strand and an alpha helix or two helices contiguous in primary sequence. In the current invention, loop sequences are typically determined with reference to the Ormo & Remington GFP structure (MMDB ID:5742) or with reference to SEQ ID NO:2 (or SEQ ID NO:8), or SEQ ID NO04. In determining the loop sequence with respect to MMDB ID:5742, the loop sequences are readily identified by those of skill in the art by visual comparison of the superimposed structures.

Heterologous peptide sequences can be inserted in any of the loops. Often, the sequences are inserted in at least two loops that are on the same face of the protein. Loops that are on the same face in SEQ ID NO:2, e.g., occur at amino acid residues 9-11, 36-40, 81-83, 114-118, 154-160, and 188-199. Another set of loops that are on the same face occur at amino acid residues 23-24, 48-56, 101-103, 128-143, 172-173, and 213-214. These loop positions in other GFP fluorescent backbone proteins can be identified by maximal sequence alignment with SEQ ID NO:2 using a sequence comparison algorithm as described herein.

Loops in a dsRED having the sequence set forth in SEQ ID NO:4 were determined by structural alignment with MMDB ID:5742. Loops on one face of dsRed are: 37-39, 75-81, 114-117, 153-156, 185-192 for the end of the barrel closest to the N and C terminii; and 22-26, 100-103, 167-170, 204-209 for the loops on the opposite end of the barrel. These loop positions in other dsRED backbone proteins can be identified by maximal sequence alignment with SEQ ID NO:4 using a sequence comparison algorithm.

The amino acid residues comprising the binding site of the fluorescent binding ligand of the invention are typically introduced into the fluorescent protein backbone within 5 amino acid residues, e.g., 5, 4, 3, 2, or 1 amino acid residue of the loop residues. Typically the binding site amino acids are inserted between residues in the loop, for example, between residues 23 and 24, 101 and 102, 172 and 173, and 213 and 214. However, a number of the fluorescent protein backbone loop residues can be substituted with the binding site, e.g., 10, 9, 8, 7, 6, 5, 4, 3, 2 or 1 amino acid may be replaced.

The peptide sequences that are inserted into the loop regions, the “binding sites” can be any number of amino acids in length. Typically, the sequences are at least 2 amino acids, and may be as large as fifty or more amino acids (antibody CDRs usually range from about 2 to about 32 amino acids). Longer sequences can also be accommodated, provided their N and C termini can be brought close together.

The sequences inserted into the loop can be from any source. Although the sequences inserted into the loop regions can be defined sequences, e.g., corresponding to the CDR regions of a known antibody, the sequences inserted into the loop regions are typically random peptide sequences or CDR sequences from many different antibodies.

In preferred embodiments, a library of fluorescent binding ligands is created in which a populations of random peptide sequences or a population of CDR sequences is generated and inserted into the loop regions. The sequences at each loop region of a particular fluorescent binding ligand is therefore typically different. Such libraries can then be screened with an antigen to identifying fluorescent binding ligands that specifically bind the antigen. Typically, libraries are generated using PCR in conjunction with other standard methodology in the art.

General Nucleic Acid Methodology

The libraries and fluorescent binding ligands of the invention are generated using basic nucleic acid methodology that is routine techniques in the field of recombinant genetics. Basic texts disclosing the general methods of obtaining and manipulating nucleic acids in this invention include Sambrook and Russell, MOLECULAR CLONING, A LABORATORY MANUAL (3rd ed. 2001) and CURRENT PROTOCOLS IN MOLECULAR BIOLOGY (Ausubel et al., eds., John Wiley & Sons, Inc. 1994-1997, 2001 version)).

Typically, the nucleic acid sequences encoding the fluorescent ligands of the invention are generated using amplification techniques. Examples of techniques sufficient to direct persons of skill through in vitro amplification methods are found in Berger, Sambrook, and Ausubel, as well as Dieffenfach & Dveksler, PCR Primers: A Laboratory Manual (1995): Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

Amplification techniques can typically be used to obtain a population of sequences, e.g., random peptide sequences or CDRs, to insert into the loop regions. In generating a population of CDR's, it is often desirable to obtain CDRs that do not include the primer sequences from the amplification primers. This can be achieved by using primers that include restriction enzyme sites, such as BpmI, that cleave at a distance from the recognition sequence. Such a method is exemplified in Example 2. The amplified population can then be introduced into the fluorescent protein backbone at the desired loop sites, for example, using appropriate adaptors and additional amplification reactions.

Random peptides can also be inserted into the loop regions of the fluorescent protein. The random peptides are inserted using methods well known in the art. For example, single-stranded, UTP-substituted DNA from a phagemid can be performed in which oligonucletides that hybridize to the sequence encoding a loop region of the fluorescent protein are used. The oligonucleotides are flanked by a region of homology, for example, 21 base pairs, on either side of the insertion site and contain random based to encode the random amino acids.

Display Libraries

Fluorescent ligand binding libraries can be constructed using a number of different display systems. In cell or virus-based systems, the ligand can be displayed, for example, on the surface of a particle, e.g., a virus or cell and screened for the ability to interact with other molecules, e.g., a library of target molecules. In vitro display systems can also be used, in which the fluorescent binding ligand is linked to an agent that provides a mechanism for coupling the fluorescent binding ligand to the nucleic acid sequence that encodes it. These technologies include ribosome display and mRNA display.

As noted above, in some instances, for example, ribosomal display, a fluorescent binding ligand is linked to the nucleic acid sequence through a physical interaction, for example, with a ribosome. In other embodiments, e.g., MRNA display, the fluorescent binding ligand may be joined to another molecule via a linking group. The linking group can be a chemical crosslinking agent, including, for example, succinimidyl-(N-maleimidomethyl)-cyclohexane-1-carboxylate (SMCC). The linking group can also be an additional amino acid sequence(s), including, for example, a polyalanine, polyglycine or similar linking group. Other near neutral amino acids, such as Ser can also be used in the linker sequence. Amino acid sequences which may be usefully employed as linkers include those disclosed in Maratea et al. (1985) Gene 40:39-46; Murphy et al. (1986) Proc. Natl. Acad. Sci. USA 83:8258-8262; U.S. Pat. Nos. 4,935,233 and 4,751,180. The linker sequence may generally be from 1 to about 50 amino acids in length, e.g., 2, 3, 4, 6, or 10 amino acids in length, but can be 100 or 200 amino acids in length.

Other chemical linkers include carbohydrate linkers, lipid linkers, fatty acid linkers, polyether linkers, e.g., PEG, etc. For example, poly(ethylene glycol) linkers are available from Shearwater Polymers, Inc. Huntsville, Ala. These linkers optionally have amide linkages, sulfhydryl linkages, or heterofunctional linkages.

Phage Display Libraries

Construction of phage display libraries exploits the bacteriophage's ability to display peptides and proteins on their surfaces, i.e., on their capsids. Often, filamentous phage such as M13, fd, or fl are used. Filamentous phage contain single-stranded DNA surrounded by multiple copies of genes encoding major and minor coat proteins, e.g., pIII. Coat proteins are displayed on the capsid's outer surface. DNA sequences inserted in-frame with capsid protein genes are co-transcribed to generate fusion proteins or protein fragments displayed on the phage surface. Phage libraries thus can display peptides representative of the diversity of the inserted sequences. Significantly, these peptides can be displayed in “natural” folded conformations. The fluorescent binding ligands expressed on phage display libraries can then bind target molecules, i.e., they can specifically interact with binding partner molecules such as antigens, e.g., (Petersen (1995) Mol. Gen. Genet. 249:425-31), cell surface receptors (Kay (1993) Gene 128:59-65), and extracellular and intracellular proteins (Gram (1993) J. Immunol. Methods 161:169-76).

The concept of using filamentous phages, such as M13 or fd, for displaying peptides on phage capsid surfaces was first introduced by Smith (1985) Science 228:1315-1317. Peptides have been displayed on phage surfaces to identify many potential ligands (see, e.g., Cwirla (1990) Proc. Natl. Acad. Sci. USA 87:6378-6382). There are numerous systems and methods for generating phage display libraries described in the scientific and patent literature, see, e.g., Sambrook and Russell, Molecule Cloning: A Laboratory Manual, 3rd edition, Cold Spring Harbor Laboratory Press, Chapter 18, 2001; “Phage Display of Peptides and Proteins: A Laboratory Manual, Academic Press, San Diego, 1996; Crameri (1994) Eur. J. Biochem. 226:53-58; de Kruif (1995) Proc. Natl. Acad. Sci. USA 92:3938-42; McGregor (1996) Mol. Biotechnol. 6:155-162; Jacobsson (1996) Biotechniques 20:1070-1076; Jespers (1996) Gene 173:179-181; Jacobsson (1997) Microbiol Res. 152:121-128; Fack (1997) J. Immunol. Methods 206:43-52; Rossenu (1997) J. Protein Chem. 16:499-503; Katz (1997) Annu. Rev. Biophys. Biomol Struct. 26:27-45; Rader (1997) Curr. Opin. Biotechnol. 8:503-508; Griffiths (1998) Curr. Opin. Biotechnol. 9:102-108.

Typically, exogenous nucleic acids encoding the protein sequences to be displayed are inserted into a coat protein gene, e.g. gene III or gene VIII of the phage. The resultant fusion proteins are displayed on the surface of the capsid. Protein VIII is present in approximately 2700 copies per phage, compared to 3 to 5 copies for protein III (Jacobsson (1996), supra). Multivalent expression vectors, such as phagemids, can be used for manipulation of the nucleic acid sequences encoding the fluorescent binding library and production of phage particles in bacteria (see, e.g., Felici (1991) J. Mol. Biol. 222:301-310).

Phagemid vectors are often employed for constructing the phage library. These vectors include the origin of DNA replication from the genome of a single-stranded filamentous bacteriophage, e.g., M13 or f1 and require the supply of the other phage proteins to create a phage. This is usually supplied by a helper phage which is less efficient at being packaged into phage particles. A phagemid can be used in the same way as an orthodox plasmid vector, but can also be used to produce filamentous bacteriophage particle that contain single-stranded copies of cloned segments of DNA.

The displayed protein does not need to be a fusion protein. For example, a fluorescent binding ligand may attach to a coat protein by virtue of a non-covalent interaction, e.g., a coiled coil binding interaction, such as jun/fos binding, or a covalent interaction mediated by cysteines (see, e.g., Crameri et al., Eur. J. Biochem. 226:53-58, 1994) with or without additional non-covalent interactions. Morphosys have described a display system in which one cysteine is put at the C terminus of the scFv or Fab, and another is put at the N terminus of g3p. The two assemble in the periplasm and display occurs without a fusion gene or protein.

The coat protein does not need to be endogenous. For example, DNA binding proteins can be incorporated into the phage/phagemid genome (see, e.g., McGregor & Robins, Anal. Biochem. 294:108-117, 2001). When the sequence recognized by such proteins is also present in the genome, the DNA binding protein becomes incorporated into the phage/phagemid. This can serve as a display vector protein. In some cases it has been shown that incorporation of DNA binding proteins into the phage coat can occur independently of the presence of the recognized DNA signal.

Other phage can also be used. For example, T7 vectors, T4 vector, T2 vectors, or lambda vectors can be employed in which the displayed product on the mature phage particle is released by cell lysis.

Another methodology is selectively infective phage (SIP) technology. which provides for the in vivo selection of interacting protein-ligand pairs. A “selectively infective phage” consists of two independent components. For example, a recombinant filamentous phage particle is made non-infective by replacing its N-terminal domains of gene 3 protein (g3p) with a protein of interest, e.g., an antigen. The nucleic acid encoding the antigen can be inserted such that it will be expressed. The second component is an “adapter” molecule in which the fluorescent ligand is linked to those N-terminal domains of g3p that are missing from the phage particle. Infectivity is restored when the displayed protein (e.g., a fluorescent biniding ligand) binds to the antigen. This interaction attaches the missing N-terminal domains of g3p to the phage display particle. Phage propagation becomes strictly dependent on the protein-ligand interaction. See, e.g., Spada (1997) J. Biol. Chem. 378:445-456; Pedrazzi (1997) FEBS Lett. 415:289-293; Hennecke (1998) Protein Eng. 11:405-410.

Other Display Libraries

In addition to phage display libraries, analogous epitope display libraries can also be used. For example, the methods of the invention can also use yeast surface displayed libraries (see, e.g., Boder, Nat. Biotechnol. 15:553-557, 1997), which can be constructed using such vectors as the pYD1 yeast expression vector. Other potential display systems include mammalian display vectors and E. coli libraries. For example, the E. coli flagellin protein can be used to display fluorescent binding ligand sequences.

In vitro display library formats known to those of skill in the art can also be used, e.g., ribosome displays libraries and mRNA display libraries. In these in vitro selection technologies, proteins are made using cell-free translation and physically linked to their encoding mRNA after in vitro translation. In typical methodology for generating these libraries, DNA encoding the sequences to be selected are transcribed in vitro and translated in a cell-free system.

In ribosome display library (see, e.g., Mattheakis et al., Proc. Natl. Acad. Sci USA 91:9022-9026, 1994; Hanes & Pluckthrun, Proc. Natl. Acad. Sci USA 94:4937-4942, 1997) the link between the mRNA encoding the fluorescent binding ligand of the invention and the ligand is the ribosome itself. The DNA construct is designed so that no stop codon is included in the transcribed mRNA. Thus, the translating ribosome stalls at the end of the mRNA and the encoded protein is not released. The encoded protein can fold into its correct structure while attached to the ribosome. The complex of mRNA, ribosome and protein is then directly used for selection against an immobilized target. The mRNA from bound ribosomal complexes is recovered by dissociation of the complexes with EDTA and amplified by RT-PCR.

Method and libraries based on mRNA display technology, also referred to herein as puromycin display display, are described, for example in U.S. Pat. Nos. 6,261,804; 6,281,223; 6207446; and 6,214553. In this technology, a DNA linker attached to puromycin is first fused to the 3′ end of mRNA. The protein is then translated in vitro and the ribosome stalls at the RNA-DNA junction. The puromycin, which mimics aminoacyl tRNA, enters the ribosomal A site and accepts the nascent polypeptide. The translated protein is thus covalently linked to its encoding mRNA. The fused molecules can then be purified and screened for binding activity. The nucleic acid sequences encoding ligands with binding activity can then be obtained, for example, using RT-PCR.

The fluorescent binding ligand and sequences, e.g., DNA linker for conjugation to puromycin, can be joined by methods well known to those of skill in the art and are described, for example, in U.S. Pat. Nos. 6,261,804; 6,281,223; 6207446; and 6,214553.

Other technologies involve the use of viral proteins (e.g., protein A) that covalently attach themselves to the genes that encodes them. Fusion proteins are created that join the fluorescent binding ligand to the protein A sequence, thereby providing a mechanims to attached the fluorescent binding ligands to the genes that encode them.

Plasmid display systems rely on the fusion of displayed proteins to DNA binding proteins, such as the lac repressor (see, e.g., Gates et al., J. Mol. Biol. 255:373-386, 1996; Methods Enzymol. 267:171-191, 1996). When the lac operator is present in the plasmid as well, the DNA binding protein binds to it and can be copurified with the plasmid. Libraries can be created linked to the DNA binding protein, and screened upon lysis of the bacteria. The desired plasmid/proteins are rescued by transfection, or amplification.

Screening Libraries

Methods of screening the libraries of the invention are well known to those in the art. The libraries are typically screened using an antigen, or molecule of interest, for which it is desirable to select a binding partner. Typically, the antigen is attached to a solid surface or a specific tag, such as biotin. The antigen (or molecule of interest) is incubated with a library of the invention. Those polypeptides that bind to the antigen are then separated from those that do not using any of a number of different methods. These methods involve washing steps, followed by elution steps. Washing can be done, for example, with PBS, or detergent-containing buffers. Elution can be performed with a number of agents, depending on the type of library. For example, an acid, a base, bacteria, or a protease can be used when the library is a phage display library.

If the library that is being screened is one in which many copies of the binding ligand are displayed on the surface of an organism (e.g., yeast or bacteria), selection can be carried out by labeling the target with a fluorescent marker (such as fluorescein) and sorting those organisms which exhibit a higher fluorescence, by virtue of their increased binding to the fluorescent target.

To facilitate the identification and isolation of the antigen-bound fluorescent ligand, the fluorescent binding ligand can also be engineered as a fusion protein to include selection markers (e.g., epitope tags). Antibodies reactive with the selection tags present in the fusion proteins or moieties that bind to the labels can then be used to isolate the antigen/fluorescent binding ligand complex via the epitope or label. For example, fluorescent binding ligand/antigen complexes can be separated from non-complexed display particle using antibodies specific for the antibody selection “tag” e.g., an SV5 antibody specific to an SV5 tag. In libraries that are constructed using a display vector, such as a phage display vector, the selected clones, e.g., phage, are then used to infect bacteria.

Other detection and purification facilitating domains include, e.g., metal chelating peptides such as polyhistidine tracts and histidine-tryptophan modules that allow purification on immobilized metals, protein A domains that allow purification on immobilized immunoglobulin, or the domain utilized in the FLAGS extension/affinity purification system (Immunex Corp, Seattle Wash.). Any epitope with a corresponding high affinity antibody can be used, e.g., a myc tag (see, e.g., Kieke (1997) Protein Eng. 10:1303-1310) or an E-tag (Pharmacia). See also Maier (1998) Anal. Biochem. 259:68-73; Muller (1998) Anal. Biochem. 259:54-61. The inclusion of a cleavable linker sequences such as Factor Xa or enterokinase (Invitrogen, San Diego Calif.) between the purification domain and binding site may be useful to facilitate purification. For example, an expression vector of the invention includes a polypeptide-encoding nucleic acid sequence linked to six histidine residues. A widely used tags is six consecutive histidine residues or 6His tag. These residues bind with high affinity to metal ions immobilized on chelating resins even in the presence of denaturing agents and can be mildly eluted with imidazole. Another exemplary epitope tag is the Selection tags can also make the epitope or binding partner (e.g., antibody) detectable or easily isolated by incorporation of, e.g., predetermined polypeptide epitopes recognized by a secondary reporter/binding molecule, e.g., leucine zipper pair sequences; binding sites for secondary antibodies; transcriptional activator polypeptides; and other selection tag binding compositions. See also, e.g., Williams (1995) Biochemistry 34:1787-1797.

The screening protocols typically employ multiple rounds of selection to identify a binding ligand with the desired properties. For example, it may be desirable to select fluorescent binding ligands with a minimum binding avidity for a target. Alternatively, a maximum binding avidity of a target may be desirable. In other uses, it may be desirable to select a fluorescent binding ligand that is thermostable at a particular temperature. For example, selection using increasingly stringent binding conditions can be used to select binding ligands that bind to a target molecule at increasingly greater binding affinities. One method of performing this selection is by decreasing concentrations of an antigen to select fluorescent binding ligands from a library that have a higher affinity for the antigen. A variety of other parameters can also be adjusted to select for high affinity binding ligands, e.g., increasing salt concentration, temperature, and the like.

Once a fluorescent ligand is selected, the nucleic acid encoding the fluorescent ligand is readily obtained. This sequence may then be expressed using any of a number of systems to obtain the desired quantities of the protein. There are many expression systems for that are well know to those of ordinary skill in the art. (See, e.g., Gene Expression Systems, Fernandes and Hoeffler, Eds. Academic Press, 1999; Ausubel, supra.) Typically, the polynucleotide that encodes the fluorescent binding ligand is placed under the control of a promoter that is functional in the desired host cell. An extremely wide variety of promoters are available, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the nucleic acids that encode the joined polypeptides are incorporated for high level expression in a desired host cell.

The invention will be further understood by the following non-limiting examples:

### EXAMPLES

### Example 1

### Identification of GFP Loop Sequences

The structure of GFP was evaluated to identify loop structures on the protein. Four loops were identified on one end of the beta-barrel of the protein at positions 23-24, 101-103, 172-173, and 213-214. These loops all faced the same direction and were selected as sites to insert CDR sequences to provide binding activity. A library of CDR3 sequences was created as set forth in Example 2.

### Example 2

### CDR3 Regions for a Fluorobody Library

This example demonstrates the generation of CDR3 sequences to be included in a fluorobody library.

In order to amplify all possible CDR3 sequences, degenerate forward or reverse primers (Table 1) were designed from an examination of the germ line sequences in V-Base (http://www.mrc-cpe.cam.ac.uk/imt-doc/restricted/ok.html). A BpmI restriction site and biotin were added to the 5′ end of each primer. Human cDNA prepared from naive lymphocytes using random hexamer primers was used as a template for PCR using all the primers in Table 1 together. 1 μl of template was amplified by PCR in 50 μl of reaction buffer containing 10 mM KCl, 20 mM Tris-HCl, pH 8.8, 2 mM MgSO₄, 10 mM (NH₂)₄ SO4, 0.1% Triton X-100, 2 U of Vent Polymerase, and 0.2 mM dNTPs using following conditions: 94° C. for 2 min, then 30 cycles of 94° C. for 30 sec, 60° C. for 30 sec, and 72° C. for 45 sec. Amplification was completed with a 10 min incubation at 72° C.

PCR products were separated in a 4% Metaphor gel (BMA, Rockland, Me.) and the population of CDR3 (ranging in size from about 75 bp to about 150 bp including primer sequences) was excised from the gel and cleaned with a gel extraction kit (Qiagen, Valencia Calif.). For all PCR amplifications, Vent, a non-error prone DNA Polymerase (New England Biolabs (NEB), Beverly Mass.) was used. This amplification protocol produced CDR3s flanked at either end by BpmI sites and Biotin.

TABLE 1

Primer sequences to amplify V_{H} CDR3 sequences:

VR35-1
5′ Biotin-CGTG CTGGAG TAT TAC TGT GCR AGA GA
VR35-2
5′ Biotin-CGTG CTGGAG TAT TAC TAT GCG AGA GA
VR35-3
5′ Biotin-CGTG CTGGAG TAT TAC TGT GCR RCA GA
VR35-4
5′ Biotin-CGTG CTGGAG TAT TAC TGT ACC ACA GA
VR35-5
5′ Biotin-CGTG CTGGAG TAT TAC TGT RCY AGA GA
VR35-6
5′ Biotin-TKTG CTGGAG TAT TAC TGT GCR AAA GA
VR35-7
5′ Biotin-TGTG CTGGAG TAT TAC TGT AAG AAA GA
VR35-8
5′ Biotin-CGTG CTGGAG TAT TAC TGT GCG AGA GG
VR33-1
5′ Biotin-CRGT CTGGAG GAC CAG GGT GCC CYG GCC
VR33-2
5′ Biotin-CGGT CTGGAG GAC CAT TGT CCC TTG GCC
VR33-3
5′ Biotin-CGGT CTGGAG GAC CAG GGT TCC TTG GCC
VR33-4
5′ Biotin-CGGT CTGGAG GAC CGT GGT CCC TTG GCC

This CDR3 population was then digested with BpmI (NEB, Beverly Mass.) at 37° C. overnight. Primer sequences conjugated to biotin were released by this digestion and removed by incubation with streptavidin magnetic beads (Dynal, Oslo Norway) for 1.5 hour at room temperature with mixing every 10 min. The beads with attached cleaved primer sequences were removed by drawing to one side in a magnetic rack, and removing the supernatant which contains the CDR3s in solution with no attached primer sequence. As BpmI cleaves to leave a 2 base pair overhang at a defined distance from its recognition site, and the primers were designed to amplify the conserved region around the highly variable CDR3, the sequence of these 2 base pair overhangs, was known. The expected overhang sequences are: 5′ CDR3-CC 3′ and 3′ TC-CDR3 5″

In order to amplify these CDR3 sequences and insert them into defined loops in GFP, adaptors were ligated to each end of the CDR3 sequences, making use of the defined overhangs described above. The adaptors consisted of portions of the GFP sequence flanking the chosen loops with the required overhangs for ligation to the CDRs. Table 2 shows the oligonucleotides representing sense or antisense of the GFP loops. Adaptor sequences are provided in the table, with the overhangs used to ligate to the CDR3 underlined and in bold. The adaptors are used in pairs as described below. One of each pair also has an overhang at the other end, designed to prevent linker ligation.

TABLE 2

adaptor sequences

Adaptor 1 (GFP 4-22)*:
5′-GGAGAAGAACTTTTCACTGGAGTTGTCCCAATTCTTGTTGAATTAGATGGTGATGTTAG^{#}-3′
3′-CCTCTTCTTGAAAAGTGACCTCAACAGGGTTAAGAACAACTTAATCTACCACTACAA-P
Adaptor 2 (GFP 24-42):
5′ P-GGGCACAAATTTTCTGTCAGAGGAGAGGGTGAAGGTGATGCTACAACGGAAAAC -3′
3′-GGCCCGTGTTTAAAAGACAGTCTCCTCTCCGACTTCCACTACGATGTTGCCTTTTGAG-5′
Adaptor 3 (GFP 85-102):
5′-AAGAGTGCCATGCCCGAAGGTTATGTACAGGAACGCACTATATCTTTCAAAGATAG-3′
3′-TTCTCACGGTA GGGCTTCCAATACATGTCCTTGCGTGATATAGAAAGTTTCTA-P-5′
Adaptor 4 (GFP 103-120):
5′-P-GACGGGACCTACAAGACGCGTGCTGAAGTCAAGTTTGAAGGTGATACCCTTG-3′
3′-GGCTGCCCTGGATGTTCTGCGCACGACTTCAGTTCAAACTTCCACTATGGGAACAA 5′
Adaptor 5 (GFP 163-172):
5′-CAAAAGAATGGAATCAAAGCTAACTTCAAAATTCGCCACAACGTTGAAG-3′
3′-TTTCTTACCTTAGTTTCGATTGAAGTTTTAAGCGGTGTTGCTTCT-P-5′
Adaptor 6 (GFP 173-184):
5′-P-GATGGTTCCGTTCAACTAGCAGACCATTATCAACAAAATACTCCAAT-3′
3′-GGCTACCAAGGCAAGTTGATCGTCTGGTAATAGTTGTTTTATGAGGTTAAC-5′
Adaptor 7 (GFP 192-213):
5′-CCTGTCCTTTTACCAGACAACCATTACCTGTCGACACAATCTGTCCTTTCGAAAGATCCCAACGAAG-3′
3′-GGACAGGAAAATGGTCTGTTGGTAATGGACAGCTGTGTTAGACAGGAAAGCTTTCTAGGGTTGCT--P-5′
Adaptor 8 (GFP 214-235):
5′- P- AAGCGTGACCACATGGTCCTTCTTGAGTTTGTAACTGCTGCTGGGATTACACATGGCATGGATG-3′
3′- GGTTCGCACTGGTGTACCAGGAAGAACTCAAACATTGACGACGACCCTAATGTGTACCGTACCTACTC-5′

*Numbers represent amino acid sequences of superfolder GFP.
^{#}Overhang nucleotides for ligation (Bold and underlined).

The 60-66 nucleotide length oligos representing sense or antisense strand of each side of the GFP loops were synthesized (Operon, Richmond, Calif.) and the 5′ site of sense of one side and antisense of the other side were phosphorylated (Table 2) so that the adaptors could ligate to the CDR3s. The oligonucleotides corresponding to each adaptor pair were mixed at 3 μm final concentration in 50 μl volume of NEB Buffer 2 (10 mM Tris-HCl, pH 7.9, 10 mM MgCl₂, 50 mM NaCl₂, and 1 mM dithiothreitol) and heated at 97° C. for 7 min to completely denature and then gradually cooled to 25° C. An aliquot of the annealed adaptor was run on a 4% metaphor gel to confirm the completion of annealing. The double-stranded oligos were mixed with the BpmI-digested CDR3 population in the presence of 40 U T4 DNA ligase and incubated at 15° C. for 16 hours in 20 μl volume of buffercontaining 50 mM Tris-HCl, pH 7.5, 10 mM MgCl₂, 10 mM DTT, 1 mM ATP, and 25 μg/ml BSA. One microliter of CDR3-GFP chimeras were further amplified using same but non-phosphorylated, oligos as primers. After these amplifications, the following fragments were created:

TABLE 3
Ligation CDR3 to oligos of GFP loops and amplification of ligated
products:

| | Assembled from fragments | | | Amplified with | |
|---|---|---|---|---|---|
| Fragment name | Fragment 1 | Fragment 2 | Fragment 3 | 5′ primer | 3′ primer |
| GFP/CDR3.1 | Adaptor 1 (GFP 4- | CDR3 | Adaptor 2 (GFP | GFP 4-22 S | GFP 24-42 AS |
| | 22) | | 24-42, S, AS | | |
| GFP/CDR3.2 | Adaptor 3 (GFP | CDR3 | Adaptor 4 (GFP | GFP 85-102 S | GFP 103-120 AS |
| | 85-102, S, AS | | 103-120, S, AS) | | |
| GFP/CDR3.3 | Adaptor 5 (GFP | CDR3 | Adaptor 6 (GFP | GFP 163-172 S | GFP 173-184 AS |
| | 163-172, S, AS) | | 173-184, S, AS) | | |
| GFP/CDR3.4 | Adaptor 7 (GFP | CDR3 | Adaptor 8 (GFP | GFP 192-213 S | GFP 214-235 AS |
| | 192-213, S, AS | | 214-235, S, AS | | |

### Example 3

### Amplification of GFP Fragments

In order to insert the CDR3s with attached GFP sequences into the GFP framework, the intervening GFP sequences were amplified. As the defined loops were 23-24, 101-102, 172-173, and 213-214, the corresponding GFP fragments were 1-25, 26-101, 102-172, 173-213, and 214-238. There is no overlap, so that these fragments alone are unable to assemble into GFP.

TABLE 4

Primers* used to amplify and create GFP fragments

| Primer name | sequence |
|---|---|
| GFP 5′ | 5′-GCAGCTGGCGCGCATGCCCTCGAGGGAGAAGAACTTTTCACTGGA-3′ |
| GFP 3′ | 5′-GAGGCGGCTAGCTTTGTAGAGCTCATCCATGCCATGTGT-3′ |
| GFP 4-22 S | 5′-GGAGAAGAACTTTTCACTGGAGTTGTCCCAATTCTTGTTGAATTAGATGGTGATGTTAG-3 |
| GFP 4-22 AS | 5′-P-AACATCACCATCTAATTCAACAAGAATTGGGACAACTCCAGTGAAAAGTTCTTC-3′ |
| GFP 25-36 | 5′-CACAAATTTTCTGTCAGAGGAGAGGGTGAAGGTGAT-3′ |
| GFP 102-91 | 5′-ATCTTTGAAAGATATAGTGCGTTCCTGTACATAACC-3′ |
| GFP 103-114 | 5′-GACGGGACCTACAAGACGCGTGCTGAAGTCAAGTTT-3′ |
| GFP 172-165 | 5′-TTCAACGTTGTGGCGAATTTTGAA-3′ |
| GFP 172-180 | 5′-TCCGTTCAACTAGCAGACCATTAT-3′ |
| GFP 213-202 | 5′-TTCGTTGGGATCTTTCGAAAGGACAGATTGTGT-3′ |
| GFP 214-235 S 5′-P-AAGCGTGACCACATGGTCCTTCTTGAGTTTGTAACTGCTGCTGGGATTACACATGGCATGGATG-3′ | |
| GFP 214-235 AS 5′-CTCATCCATGCCATGTGTAATCCCAGCAGCAGTTACAAACTCAAGAAGGACCATGTGGTCACGCTTGG-3′ | |

*These primers are different from the oligos used for ligation and assembling of GFP. They are specifically designed to efficiently amplify GFP fragments. They are also not phosphorylated.

Super folder GFP was cloned into pDAN5, a phagemid display vector. The ability of this version of GFP to be displayed on phage demonstrated that displaying a binding ligand library using GFP as a scaffold was feasible.

To minimize the parental GFP background, two different constructs were prepared: pDAN5 GFP 1-202 and pDAN5 GFP 25-238. Neither had intrinsic fluorescence activity. Therefore, when used as templates to produce the fragments described above, there was no possibility that the library could become contaminated with full-length fluorescent GFP. With the exception of the first fragment, GFP(4-22) and the last fragment, GFP(213-235), which were created by annealing of two oligonucleotides, the GFP fragments were amplified with paired primers as described in table 5.

TABLE 5
Assembly of GFP fragments

| Fragment name | 5′ PCR primer | 3′ PCR primer | Template |
|---|---|---|---|
| GFP(4-22) | GFP 4-22 S | GFP 4-22 AS | Oligo assembly |
| GFP(25-102) | GFP 25-36 | GFP 102-91 | pDAN5 GFP 1-202 |
| GFP(103-172) | GFP 103-114 | GFP 172-165 | pDAN5 GFP1-202 |
| GFP(175-213) | GFP 175-182 | GFP 213-201 | pDAN5 GFP25-238 |
| GFP(214-235) | GFP 214-235 S | GFP 214-235 AS | Oligo assembly |

PCR amplification conditions were 94° C. for 2 min initial denaturation followed by 30 cycles of 94° C. denaturation for 1 min, 60° C. annealing (annealing temperature for fragment 101-172 was 52° C.) for 1 min, and 72° C. extension for 2 min in 50 μl volumes of Vent Polymerase buffer (10 mM KCl, 20 mM Tris-HCl, pH 8.8, 2 mM MgSO₄, 10 mM (NH₂)₄ SO4, 0.1%Triton X-100, 2 U of Vent Polymerase, and 0.2 mM dNTPs). Heating at 72° C. for 10 min completed the amplification reactions. The desired sizes of PCR products were excised from a gel and cleaned with Gel Extraction Kit (Qiagen, Valencia Calif.).

Example 4

### Assembly of GFP-CDR Fragments

This example demonstrates the assembly of the GFP-CDR fragments to generate a fluorobody library. GFP fragment (200 μg) and 400 μg of GFP-CDR3 fragments (purified from 1.5% Metaphor gel) were mixed with PCR reagents in 50 μl of Vent Polymerase buffer including GFPs. Amplification was performed at 94° C. for 5 min followed by 25 cycles of 94° C. 1 min, 58° C. for 1.30 min, and 72° C. for 2 min and 10 min additional incubation at 72° C. During the first 5 cycles, no primers were added, thereby allowing assembly to occur.

After this, the reaction was paused at 94° C. to add primers. Assembly was carried out in a number of rounds. In the first round, the fragments in Table 5 were generated by using the primers and fragments shown in Table 6. These were in turn used to further assemble the fragments described in Tables 6 and 7 using the same conditions as described above.

TABLE 6

| First Round | Assembled from fragments | | | Amplified with | |
|---|---|---|---|---|---|
| Fragment name | Fragment 1 | Fragment 2 | Fragment 3 | 5′ primer | 3′ primer |
| GFP(4-CDR3-101) | GFP (4-22) | GFP/CDR3.1 | GFP(25-102) | GFP 5′ | GFP 102-91 |
| GFP(25-GDR3-172) | GFP (25-102) | GFP/CDR3.2 | GFP (103-172) | GFP 25-36 | GFP 172-165 |
| GFP(103-CDR3-213) | GFP (103-172) | GFP/CDR3.3 | GFP (175-213) | GFP 103-114 | GFP 213-202 |
| GFP(173-CDR3-235) | GFP (175-213) | GFP/CDR3.4 | GFP (214-235) | GFP 172-180 | GFP 3′ |

TABLE 7

| Second Round | Assembled from fragments | | Amplified with | |
|---|---|---|---|---|
| Fragment name | Fragment 1 | Fragment 2 | 5′ primer | 3′ primer |
| GFP(4-CDR3₂-172) | GFP(4-CDR3-101) | GFP(25-CDR3-172) | GFP 5′ | GFP 172-165 |
| GFP (103-CDR3₂-235) | GFP (103-CDR3-213) | GFP (173-CDR3-235) | GFP 103-114 | GFP 3′ |

TABLE 8

| | | |
|---|---|---|
| Third Round | Assembled from fragments | Amplified with |

| Fragment name | Fragment 1 | Fragment 2 | 5′ primer | 3′ primer |
|---|---|---|---|---|
| Fluorobody | GFP (4-CDR3₂-172) | GFP (103-CDR32-235) | GFP 5′ | GFP 3′ |

The final library of fluorobody genes was cut with NheI and BssHII, gel purified, and cloned into pDAN5, a phagemid display vector, cut with the same enzymes and gel purified.

The final number of independent clones was about 10,000,000, of which about 60% were fluorescent.

### Example 5

### Screening a Fluorobody Library with a Protein Antigen

The phage displayed fluorobody library was selected against the following antigens: myoglobin, IgG, human serum albumin, frequenin, phosphorylase B, alcohol dehydrogenase, and ubiquitin. Screening was performed using 96-well immunopins that were coated with 100 μl of protein at 10 μg/ml in PBS overnight at 4° C., and subsequently blocked with 200 μl of 3% BSA for two hours at room temperature. Pins were further incubated with 100 μl of 10¹⁰ phage/ml for 2 hours at room temperature. Following 6 washes with PBS-Tween (0.05%), and PBS alone, phages were eluted with 100 μl of 0.1 M HCl, then neutralized with Tris-HCl, pH8. Phage were amplified overnight by infection in SS330 or XL-blue E. coli suppressor strains at 37° C. Three rounds of selection were carried out and fluorescent green colonies were manually picked. Specificity was tested using specific and non-specific proteins.

The clones were further sequenced to identify inserted CDR3 sequences. The green fluorescent colonies typically contained CDRs, whereas the white colonies, i.e., non-fluorescent colonies, contained longer, non-CDR domains (derived from the RT-PCR of non-CDR mRNAs that included frame-shifts, etc.

### Example 6

### Generation of a GFP library with random nucleotides.

This library was generated using standard techniques. Briefly, single-stranded UTP DNA was made by transfecting the pDAN5-GFP plasmid into E. coli CJ236, preparing phagemid particles from a single colony, and purifying the single-stranded, UTP-substituted DNA. The mutagenesis reaction was carried out using four oligonucleotides that hybridize to the same sites described in Example 3. The oligonucleotides were flanked by 21 bp homology on either side of the insertion site and contained 9 random bases in the format NNKNNKNNK, encoding 3 random amino acids. Approximately 40% of the library was fluorescent.

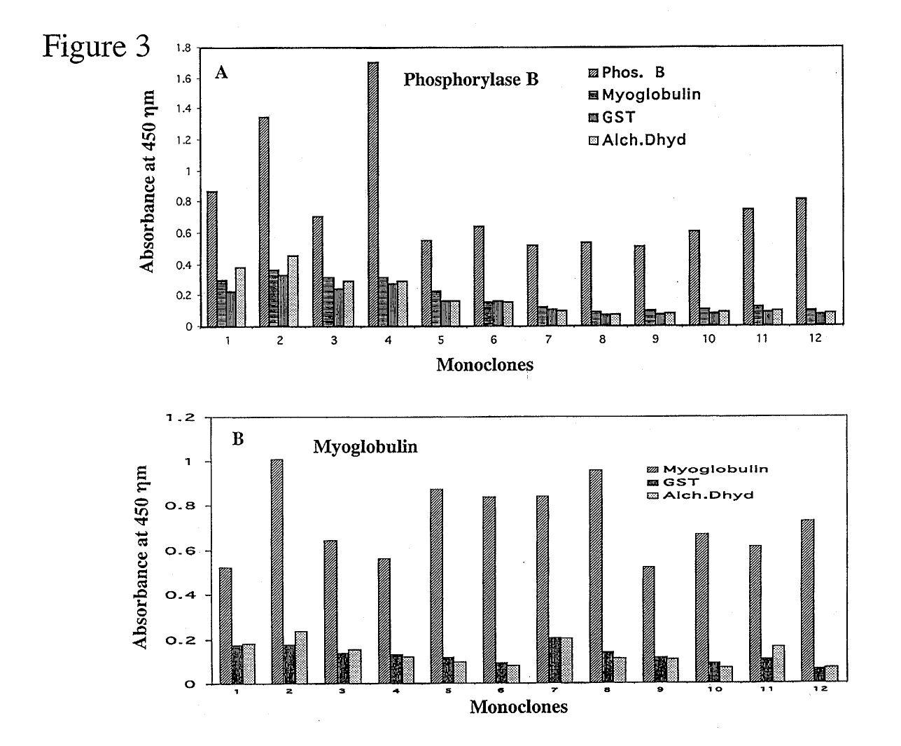

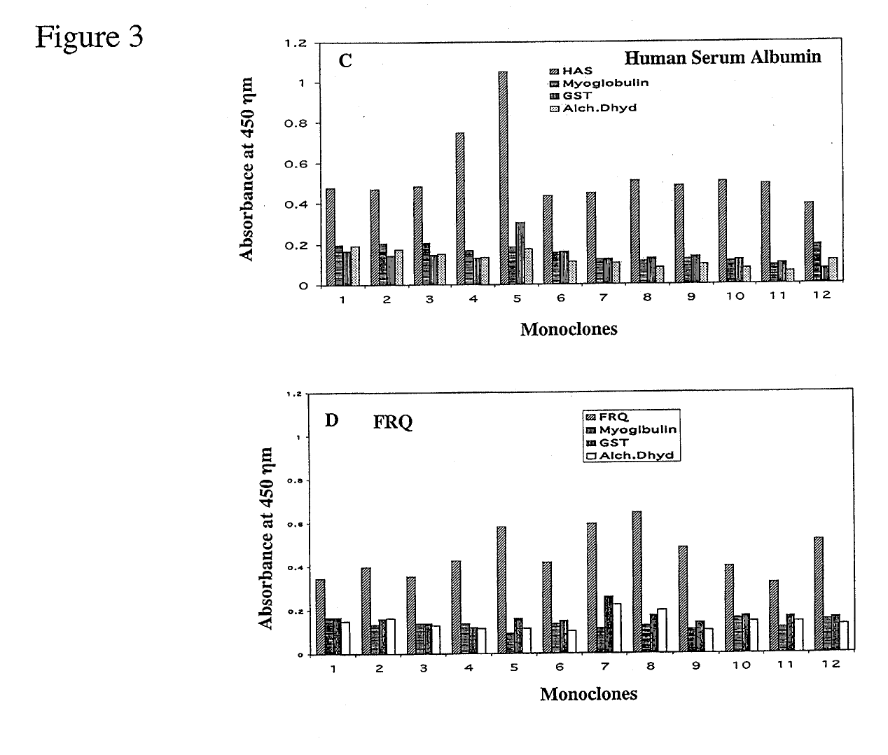

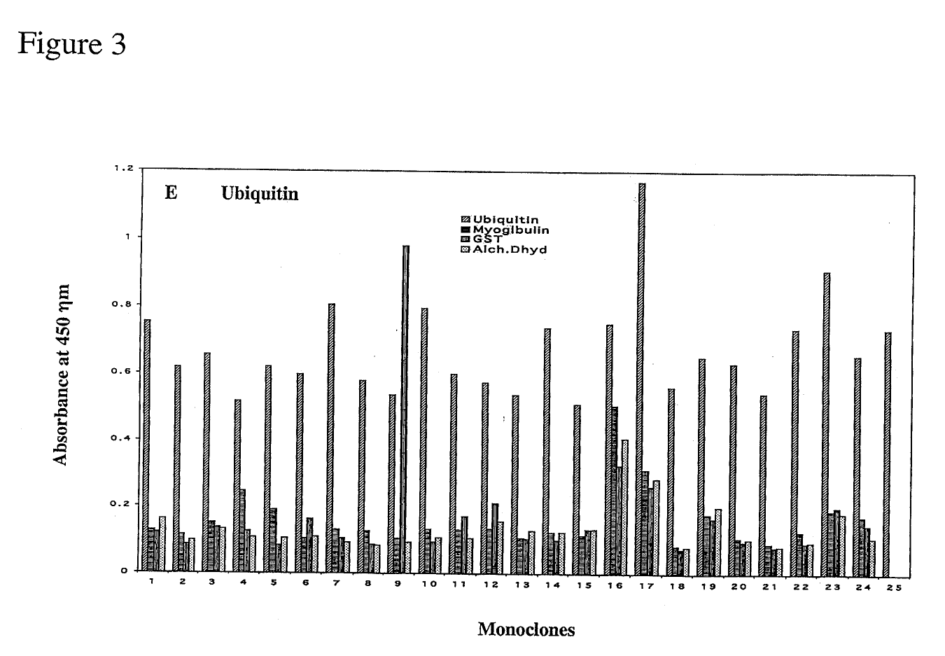

Specific fluorobodies were selected against all antigens tested (ubiquitin, human serum albumin, myoglobulin, and frequenin). After selection, individual monoclonal fluorobodies were tested for binding to both specific and non-specific targets by ELISA in a sandwich assay in which specific or non-specific antigen was bound to plastic ELISA plates. After blocking the plates with milk to prevent non-specific binding, fluorobody phage or soluble fluorobodies were added to the specific or non-specific antigens. Phage fluorobodies were detected with labeled anti-phage antibody, while soluble fluorobodies were detected with an SV5 antibody, which specifically binds to the SV5 tag present at the C-terminus of the fluorobody, and labeled anti-mouse serum. The absorbances for specific and non-specific binding are indicated in FIG. 3 and summarized in Table 9. Almost all fluorobodies were specific for their targets without any recognition of irrelevant targets.

TABLE 9
Mean absorbances of clones detected against specific and non-specific
targets

| Targets | Positive^{a} | Abs/Specific^{b} | Abs/Non-Specific^{c} |
|---|---|---|---|
| Ubiquitin | 25 of 92 | 0.654 ± 0.140 | 0.125 ± 0.037 |
| FRQ | 12 of 92 | 0.459 ± 0.096 | 0.147 ± 0.032 |
| Human S A | 9 of 92 | 0.553 ± 0.169 | 0.143 ± 0.045 |
| Myoglobin | 10 of 92 | 0.766 ± 0.163 | 0.126 ± 0.067 |
| Phosphorylase B | 8 of 92 | 0.857 ± 0.414 | 0.213 ± 0.102 |

^{a}clones with ELISA signal three times greater than control levels are considered positive
^{b}mean absorbance of positive clones
^{c}mean absorbance of ELISA performed against three non-specific proteins

All publications, patents, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

TABLE OF SEQUENCES

GFP folder variant nucleic acid sequence

SEQ ID NO:1

ATGAGCAAAGGAGAAGAACTTTTCACTGGAGTTGTCCCAATTCTTGTTGA
ATTAGATGGTGATGTTAATGGGCACAAATTTTCTGTCAGTGGAGAGGGTG
AAGGTGATGCTACATACGGAAAACTCACCCTTAAATTTATTTGCACTACT
GGAAAACTACCTGTTCCATGGCCAACACTTGTCACTACTCTGACCTATGG
TGTTCAATGCTTTTCCCGTTATCCGGATCACATGAAACGGCATGACTTTT
TCAAGAGTGCCATGCCCGAAGGTTATGTACAGGAACGCACTATATCTTTC
AAAGATGACGGGAACTACAAGACGCGTGCTGAAGTCAAGTTTGAAGGTGA
TACCCTTGTTAATCGTATCGAGTTAAAAGGTATTGATTTTAAAGAAGATG
GAAACATTCTCGGACACAAACTCGAGTACAACTATAACTCACACAATGTA
TACATCACGGCAGACAAACAAAAGAATGGAATCAAAGCTAACTTCAAAAT
TCGCCACAACATTGAAGATGGTTCCGTTCAACTAGCAGACCATTATCAAC
AAAATACTCCAATTGGCGATGGCCCTGTCCTTTTACCAGACAACCATTAC
CTGTCGACACAATCTGCCCTTTCGAAAGATCCCAACGAAAAGCGTGACCA
CATGGTCCTTCTTGAGTTTGTAACTGCTGCTGGGATTACACATGGCATGG
ATGAGCTCTACAAATAA

GFP folder variant amino acid sequence

SEQ ID NO:2

MSKGEELFTGVVPILVELDGDVNGHKFSVSGEGEGDATYGKLTLKFICTT
GKLPVPWPTLVTTLTYGVQCFSRYPDHMKRHDFFKSAMPEGYVQERTISF
KDDGNYKTRAEVKFEGDTLVNRIELKGIDFKEDGNILGHKLEYNYNSHNV
YITADKQKNGIKANFKIRHNIEDGSVQLADHYQQNTPIGDGPVLLPDNHY
LSTQSALSKDPNEKRDHMVLLEFVTAAGITHGMDELYK*

dsRED nucleotide sequence:

SEQ ID NO:3

ATGAGGTCTTCCAAGAATGTTATCAAGGAGTTCATGAGGTTTAAGGTTCG
CATGGAAGGAACGGTCAATGGGCACGAGTTTGAAATAGAAGGCGAAGGAG
AGGGGAGGCCATACGAAGGCCACAATACCGTAAAGCTTAAGGTAACCAAG
GGGGGACCTTTGCCATTTGCTTGGGATATTTTGTCACCACAATTTCAGTA
TGGAAGCAAGGTATATGTCAAGCACCCTGCCGACATACCAGACTATAAAA
AGCTGTCATTTCCTGAAGGATTTAAATGGGAAAGGGTCATGAACTTTGAA
GACGGTGGCGTCGTTACTGTAACCCAGGATTCCAGTTTGCAGGATGGCTG
TTTCATCTACAAGGTCAAGTTCATTGGCGTGAACTTTCCTTCCGATGGAC
CTGTTATGCAAAAGAAGACAATGGGCTGGGAAGCCAGCACTGAGCGTTTG
TATCCTCGTGATGGCGTGTTGAAAGGAGAGATTCATAAGGCTCTGAAGCT
GAAAGACGGTGGTCATTACCTAGTTGAATTCAAAAGTATTTACATGGCAA
AGAAGCCTGTGCAGCTACCAGGGTACTACTATGTTGACTCCAAACTGGAT
ATAACAAGCCACAACGAAGACTATACAATCGTTGAGCAGTATGAAAGAAC
CGAGGGACGCCACCATCTGTTCCTTTAA

dsRED Amino acid sequence:

SEQ ID NO:4

MRSSKNVIKEFMRFKVRMEGTVNGHEFEIEGEGEGRPYEGHNTVKLKVTK
GGPLPFAWDILSPQFQYGSKVYVKHPADIPDYKKLSFPEGFKWERVMNFE
DGGVVTVTQDSSLQDGCFIYKVKFIGVNFPSDGPVMQKKTMGWEASTERL
YPRDGVLKGEIHKALKLKDGGHYLVEFKSIYMAKKPVQLPGYYYVDSKLD
ITSHNEDYTIVEQYERTEGRHHLFL

superfolder GFP nucleic acid sequence

SEQ ID NO:5

ATGAGCAAAGGAGAAGAACTTTTCACTGGAGTTGTCCCAATTCTTGTTGA
ATTAGATGGTGATGTTAATGGGCACAAATTTTCTGTCAGaGGAGAGGGTG
AAGGTGATGCTACAaACGGAAAACTCACCCTTAAATTTATTTGCACTACT
GGAAAACTACCTGTTCCATGGCCAACACTTGTCACTACTCTGACCTATGG
TGTTCAATGCTTTTCCCGTTATCCGGATCACATGAAACGGCATGACTTTT
TCAAGAGTGCCATGCCCGAAGGTTATGTACAGGAACGCACTATATCTTTC
AAAGATGACGGGAcCTACAAGACGCGTGCTGAAGTCAAGTTTGAAGGTGA
TACCCTTGTTAATCGTATCGAGTTAAAAGGTATTGATTTTAAAGAAGATG
GAAACATTCTCGGACACAAACTCGAGTACAACTtTAACTCACACAATGTA
TACATCACGGCAGACAAACAAAAGAATGGAATCAAAGCTAACTTCAAAAT
TCGCCACAACgTTGAAGATGGTTCCGTTCAACTAGCAGACCATTATCAAC
AAAATACTCCAATTGGCGATGGCCCTGTCCTTTTACCAGACAACCATTAC
CTGTCGACACAATCTGtCCTTTCGAAAGATCCCAACGAAAAGCGTGACCA
CATGGTCCTTCTTGAGTTTGTAACTGCTGCTGGGATTACACATGGCATGG
ATGAGCTCTACAAATAA

superfolder GFP nucleic acid sequence

SEQ ID NO:6

MSKGEELFTGVVPILVELDGDVNGHKFSVRGEGEGDATNGKLTLKIFICT
TGKLPVPWPTLVTTLTYGVQCFSRYPDHMKRHDFFKSAMPEGYVQERTIS
FKDDGTYKTRAEVKFEGDTLVNRIELKGIDFKEDGNILGHKLEYNFNSHN
VYITADKQKNGIKANFKIRHNVEDGSVQLADHYQQNTPIGDGPVLLPDNH
YLSTQSVLSKDPNFKRDHMVLLEFVTAAGITHGMDELYK*

Wildtype GFP nucleic acid sequence,
protein encoding region
from Genbank accession number M62653

SEQ ID NO:7

ATGAGTAAAGGAGAAGAACTTTTCACTGGAGTTGTCCCAATTCTTGTTGA
ATTAGATGGTGATGTTAATGGGCACAAATTTTCTGTCAGTGGAGAGGGTG
AAGGTGATGCAACATACGGAAAACTTACCCTTAAATTTATTTGCACTACT
GGAAAACTACCTGTTCCATGGCCAACACTTGTCACTACTTTCTCTTATGG
TGTTCAATGCTTTTCAAGATACCCAGATCATATGAAACAGCATGACTTTT
TCAAGAGTGCCATGCCCGAAGGTTATGTACAGGAAAGAACTATATTTTTC
AAAGATGACGGGAACTACAAGACACGTGCTGAAGTCAAGTTTGAAGGTGA
TACCCTTGTTAATAGAATCGAGTTAAAAGGTATTGATTTTAAAGAAGATG
GAAACATTCTTGGACACAAATTGGAATACAACTATAACTCACACAATGTA
TACATCATGGCAGACAAACAAAAGAATGGAATCAAAGTTAACTTCAAAAT
TAGACACAACATTGAAGATGGAAGCGTTCAACTAGCAGACCATTATCAAC
AAAATACTCCAATTGGCGATGGCCCTGTCCTTTTACCAGACAACCATTAC
CTGTCCACACAATCTGCCCTTTCGAAAGATCCCAACGAAAAGAGAGACCA
CATGGTCCTTCTTGAGTTTGTAACAGCTGCTGGGATTACACATGGCATGG
ATGAACTATACAAATAA

Wildtype GFP amino acid sequence
encoded by SEQ ID NO:7
(Swiss protein database accession P42212)

SEQ ID NO:8

MSKGEELFTGVVPILVELDGDVNGHKFSVSGEGEGDATYGKLTLKFICTT
GKLPVPWPTLVTTFSYGVQCFSRYPDHMKQHDFFKSAMPEGYVQERTIFF
KDDGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDNHY
LSTQSALSKDPNEKRDHMVLLEFVTAAGITHGMDELYK

What is claimed is:

1. A binding ligand with intrinsic fluorescence comprising a fluorescent protein that has a structure with a root mean square deviation of less than 5 angstroms from the 11 strand beta barrel structure of the green fluorescent protein (GFP) structure MMDB Id: 5742; wherein the fluorescent protein comprises heterologous binding sites in at least two loop positions on the surface of the fluorescent protein; and has fluorescent activity.

2. The binding ligand of claim 1, wherein the fluorescent protein has increased folding ability in comparison to a protein having the sequence of SEQ ID NO:2 or SEQ ID NO:4.

3. The binding ligand of claim 1, wherein the two loop positions are on the same face of the protein.

4. The binding ligand of claim 3, wherein the loop positions are within 5 amino acids of the positions selected from the group consisting of positions 9-11, 36-40, 81-83, 114-118, 154-160, and 188-199 as determined by maximal correspondence to SEQ ID NO:2.

5. The binding ligand of claim 3, wherein the loop positions are within 5 amino acids of the positions selected from the group consisting of positions 23-24, 48-56, 101-103, 128-143, 172-173, and 213-214 as determined by maximal correspondence to SEQ ID NO:2.

6. The binding ligand of claim 3, wherein the loop positions are within 5 amino acids of the positions selected from the group consisting of positions 37-39, 75-81, 114-117, 153-156, 185-192 as determined by maximal correspondence to SEQ ID NO:4.

7. The binding ligand of claim 3, wherein the loop positions are within 5 amino acids of the positions selected from the group consisting of positions 22-26, 100-103, 167-170, and 204-209 as determined by maximal correspondence to SEQ ID NO:4.

8. The binding ligand of claim 1, wherein the binding sites comprise random peptides.

9. The binding ligand of claim 1, wherein the binding sites comprises complementarity determining regions (CDRs).

10. The binding ligand of claim 1, wherein the binding ligand comprises heterologous binding sites at three loop regions.

11. The binding ligand of claim 1, wherein the binding ligand comprises heterologous binding sites at four loop regions.

12. The binding ligand of claim 2, wherein the fluorescent protein has the sequence set forth in SEQ ID NO:5.

13. An expression vector comprising a nucleic acid sequence encoding a fluorescent binding ligand as set forth in claim 1.

14. A host cell comprising the expression vector of claim 13.

15. A library of fluorescent binding ligands as set forth in claim 1.

16. A library comprising a population of nucleic acid sequences encoding fluorescent binding ligands as set forth in claim 1.

17. A library of claim 16, wherein the nucleic acid sequence encoding the fluorescent binding ligand is linked to a polypeptide selected from the group consisting of a phage coat polypeptide, a bacterial outer membrane protein, a yeast outer membrane protein, and a DNA binding protein.

18. A library of claim 16, wherein the library is a display library.

19. A library of claim 18, wherein the library is a phage display library.

20. A library of claim 18, wherein the library is a ribosomal display library.

21. A library of claim 18, wherein the library is an mRNA display library.

22. A library of claim 18, wherein the library is a bacterial display library.

23. A library of claim 18, wherein the library is a plasmid display library.

24. A library of claim 18, wherein the library is a yeast display library.

25. A method of preparing a binding ligand with intrinsic fluorescence that binds to a target antigen, the method comprising providing a fluorescent protein that has a structure with a root mean square deviation of less than 5 angstroms from the 11 strand beta barrel structure of the green fluorescent protein (GFP) structure MMDB Id: 5742; and inserting a heterologous binding site into at least two loop regions on the surface of the protein, thereby obtaining a binding ligand with intrinsic fluorescence.

26. The method of claim 25, wherein the two loop regions are on the same face of the protein.

27. The method of claim 26, wherein the loop positions are within 5 amino acids of the positions selected from the group consisting of positions 9-11, 36-40, 81-83, 114-118, 154-160, and 188-199 as determined by maximal correspondence to SEQ ID NO:2.

28. The method of claim 26, wherein the loop positions are within 5 amino 2 acids of the positions selected from the group consisting of positions 23-24, 48-56, 101-103, 128-143, 172-173, and 213-214 as determined by maximal correspondence to SEQ ID NO:2.

29. The method of claim 26, wherein the loop positions are within 5 amino acids of the positions selected from the group consisting of positions 37-39, 75-81, 114-117, 153-156, 185-192 as determined by maximal correspondence to SEQ ID NO:4.

30. The method of claim 26, wherein the loop positions are within 5 amino acids of the positions selected from the group consisting of positions 22-26, 100-103, 167-170, and 204-209 as determined by maximal correspondence to SEQ ID NO:4.

31. The method of claim 25, wherein the binding sites comprise random peptides.

32. The method of claim 25, wherein the binding sites comprises complementarity determining regions (CDRs).

33. The method of claim 25, wherein the binding ligand comprises binding sites at three loop regions.

34. The method of claim 25, wherein the binding ligand comprises binding sites at four loop regions.

35. The method of claim 25, wherein the fluorescent protein has increased folding ability in comparison to a protein having the sequence of SEQ ID NO:2.

36. The method of claim 35, wherein the fluorescent protein has the sequence set forth in SEQ ID NO:5.

37. A method of identifying a binding ligand with intrinsic fluorescence that specifically binds to a target molecule, the method comprising:
providing a library as set forth in claim 16;
screening the library with the target molecule; and
selecting a binding ligand that binds to the target molecule.
